(19) United States
(12) Patent Application Publication
(10) Pub. No.: US 2018/0372076 A1
(43) Pub. Date: Dec. 27, 2018

(54) SYRINGE ACTUATION SYSTEM

(71) Applicant: SHINANO KENSHI KABUSHIKI KAISHA, Nagano (JP)

(72) Inventor: Koichi ICHIKI, Torrance, CA (US)

(21) Appl. No.: 16/003,582

(22) Filed: Jun. 8, 2018

Related U.S. Application Data
(60) Provisional application No. 62/525,119, filed on Jun. 26, 2017.

(51) Int. Cl. F03G 7/06; A61M 5/315

(52) U.S. Cl. CPC F03G 7/065; A61M 5/31576; A61M 2205/0288; A61M 2005/31588; A61M 2205/0266

(57) ABSTRACT

An actuator apparatus is provided. The actuator apparatus includes a slidable plunger with a remote end located proximate a reservoir within a vessel having an exit opening, a shape memory alloy (SMA) coil wound around the slidable plunger, and electronic circuitry connected to the SMA coil. Liquid may be provided to the reservoir via the exit opening, thereby sliding the slidable plunger away from the exit opening and expanding the SMA coil. Wherein when the SMA coil is expanded, electricity is provided via the electronic circuitry to the SMA coil to cause the SMA coil to return to a compressed configuration, moving slidable plunger within the vessel to force the liquid from the vessel via the exit opening.

### BACKGROUND OF THE INVENTION

### Field of the Invention

The present invention generally relates to actuation devices, and more particularly to devices employed to efficiently and effectively actuate syringes or syringe-type devices without the need for manual operation.

### Description of the Related Art

Many applications employ syringes or syringe type devices to distribute or provide liquid. Applications of syringe type devices include medical applications, wherein a liquid drug is received and transmitted, as well as collecting and distributing liquids in the food industry, pharmaceutical industry, in the collection of samples, and almost anywhere a limited or measured amount of liquid is desired to be taken from one location and placed in or on another.

Drawbacks with syringes include the need for human interaction. In cases where mechanical syringes are employed, a syringe pump or activation device is sometimes driven automatically, such as using a lead screw, reduction gears, and electric motors. Such devices can be expensive and cumbersome, and failure of these devices can be highly problematic in that the line or syringe operation must be shut down to repair or replace the failing device.

Existing syringe drive mechanisms include lead screws. One design is presented in U.S. Pat. No. 6,656,158, wherein a threaded lead screw is received in a reservoir and a plunger has an outer periphery linearly slidable along a side wall of the reservoir and an inner periphery threadedly received on the lead screw. The plunger in such a device is non-rotatable with respect to the side wall such that rotating the lead screw causes the plunger to advance within the reservoir and force fluid through the outlet. Again, such a design tends to be expensive and include a number of parts that can wear or break.

Further, currently available syringe actuation devices are set for a specific syringe size and the components and sizing cannot be changed readily. If a different size syringe is required, the entire arrangement typically needs to be redesigned and/or all components completely replaced. In particular, downsizing may be prohibited in that certain components may not be fabricated small enough to collect and distribute a small quantity of fluid. Additionally, these prior designs employing a lead screw, or other screw based device tend to be fairly slow in their actuation, which is undesirable in a number of applications. Such designs can be beneficial for continuous injection at low speed, but are not optimal when a rapid single injection is desired.

It would therefore be beneficial to provide a syringe type actuation arrangement that addresses issues with previous designs, is light, efficient, portable, and self-contained, can operate relatively quickly and can address small size liquid actuation needs.

### SUMMARY OF THE INVENTION

According to one aspect of the present design, there is provided an actuator apparatus, comprising a slidable plunger comprising a remote end located proximate a reservoir within a vessel having an exit opening, a shape memory alloy (SMA) coil wound around the slidable plunger, and electronic circuitry connected to the SMA coil. Liquid may be provided to the reservoir via the exit opening, thereby sliding the slidable plunger away from the exit opening and expanding the SMA coil. When the SMA coil is expanded, electricity is provided via the electronic circuitry to the SMA coil to cause the SMA coil to return to a compressed configuration, moving the slidable plunger within the vessel to force the liquid from the vessel via the exit opening.

According to a second aspect, there is provided an actuator apparatus, comprising a slidable actuation member located within a vessel having an exit opening, wherein the slidable actuation member positioned a distance from the exit opening forms a reservoir within the vessel, a shape memory alloy (SMA) coil positioned in association with the slidable actuation member to move the slidable actuation member within the vessel, and electronic circuitry connected to the SMA coil. Liquid may be provided to the reservoir via the exit opening, thereby moving the slidable actuation member away from the exit opening and compressing the SMA coil, and wherein when the SMA coil is thus compressed, electricity is provided via the electronic circuitry to the SMA coil to cause the SMA coil to return to an extended shape, pushing the slidable actuation member and expelling liquid from the vessel via the exit opening.

According to a further aspect, there is provided an actuation method, comprising providing liquid through an exit opening to a reservoir in a vessel, thereby sliding a slidable actuation member located within the vessel away from the exit opening and concurrently compressing a shape memory alloy (SMA) coil positioned in association with the slidable actuation member, and applying electricity to the SMA coil via electronic circuitry. Applying electricity results in a production of heat, transitioning the SMA coil to an extended shape, thereby pushing liquid from the reservoir by pushing the slidable actuation member and expelling liquid through the exit opening.

These and other advantages of the present invention will become apparent to those skilled in the art from the following detailed description of the invention and the accompanying drawings.

### BRIEF DESCRIPTION OF THE DRAWINGS

For a more complete understanding of the present disclosure, reference is now made to the following figures, wherein like reference numbers refer to similar items throughout the figures:

FIG. 1 is a generalized representation of an actuation device in accordance with the present design including the SMA coil;

FIG. 2 illustrates an actuation device with fluid being provided through an opening to a reservoir inside a vessel;

FIG. 3 shows the reservoir filled and the SMA coil expanded;

FIG. 4 shows power being applied to the SMA coil and the coil contracting to the shape shown, expelling liquid;

FIG. 5 illustrates a second embodiment with a plunger element fixed to the SMA coil within the vessel;

FIG. 6 is another view of the second embodiment;

FIG. 7 is a view of power being applied to the SMA coil and the SMA coil retracting to draw in liquid;

FIG. 8 is a view of the retracted SMA coil with a reservoir within the vessel filled with liquid;

FIG. 9 shows the size of the SMA coil and a plunger in one embodiment of the design;

FIG. 10 illustrates the SMA coil with the plunger having a cylindrical tube base;

FIG. 11 shows the SMA coil connected to electrical circuitry, here electrical clips, with a representation of the plunger and SMA coil in an expanded orientation; and

FIG. 12 illustrates the SMA coil connected to electrical circuitry in the form of electrical clips in this view, with a representation of the plunger and SMA coil in an compressed orientation.

The exemplification set out herein illustrates particular embodiments, and such exemplification is not intended to be construed as limiting in any manner.

### DETAILED DESCRIPTION OF THE DISCLOSURE

The following description and the drawings illustrate specific embodiments sufficiently to enable those skilled in the art to practice the system and method described. Other embodiments may incorporate structural, logical, process and other changes. Examples merely typify possible variations. Individual components and functions are generally optional unless explicitly required, and the sequence of operations may vary. Portions and features of some embodiments may be included in or substituted for those of others.

The present design is a syringe or syringe type actuation device that employs an SMA (shape memory alloy) coil primarily used in small syringe actuation situations. Such a device enables the original shape of the SMA coil to be retained and when deformed, returned to its original shape when heated.

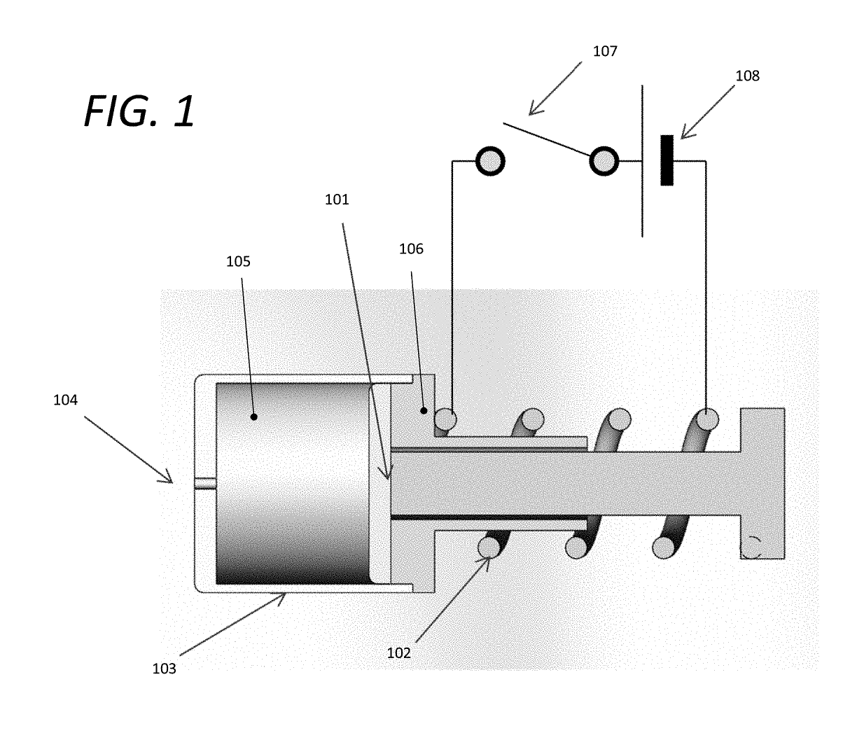

FIG. 1 discloses an arrangement according to the present design, including plunger 101 positioned within cylindrical tube 103. Cylindrical tube 103 also includes liquid 105. Housing 106 is provided in this arrangement outside plunger 101 and meets/fits with cylindrical tube 103. Also provided is SMA coil 102 on the outside of housing 106 and plunger 101, and contacting the right end of cylindrical tube 103 in this view. SMA coil 102 in this embodiment is connected to switch 107 and power source 108.

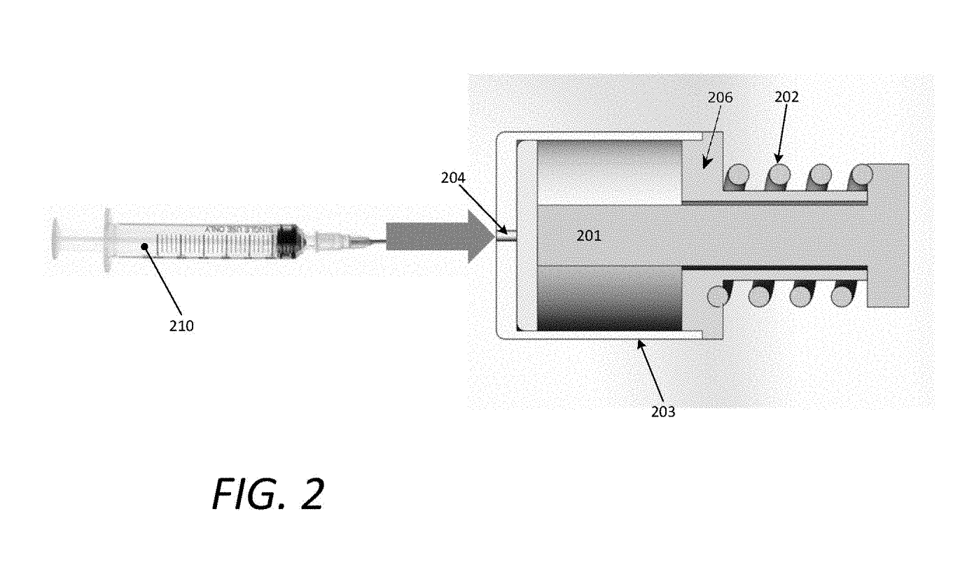

The arrangement of FIG. 1 operates as follows when used for pumping liquid. Liquid is drawn in from nozzle 104 by retracting plunger 101, i.e. drawing plunger 101 away from cylindrical tube 103. This is shown in FIG. 2, where a syringe 210 is shown providing fluid via nozzle 204 by drawing the fluid using plunger 201. In this view, SMA coil 202 is relatively compressed, and cylindrical tube 203 is shown. Cylindrical tube 203 may also be considered a vessel able to maintain a reservoir for liquid when the plunger 201 is retracted. The syringe 210 is used to provide a liquid, such as a liquid drug, into the reservoir so formed, wherein the filling pressure deforms SMA coil 202. In this arrangement, plunger 201 is surrounded by housing 206. Electricity and/or heat is applied when desired to expel the liquid drug from the nozzle 204.

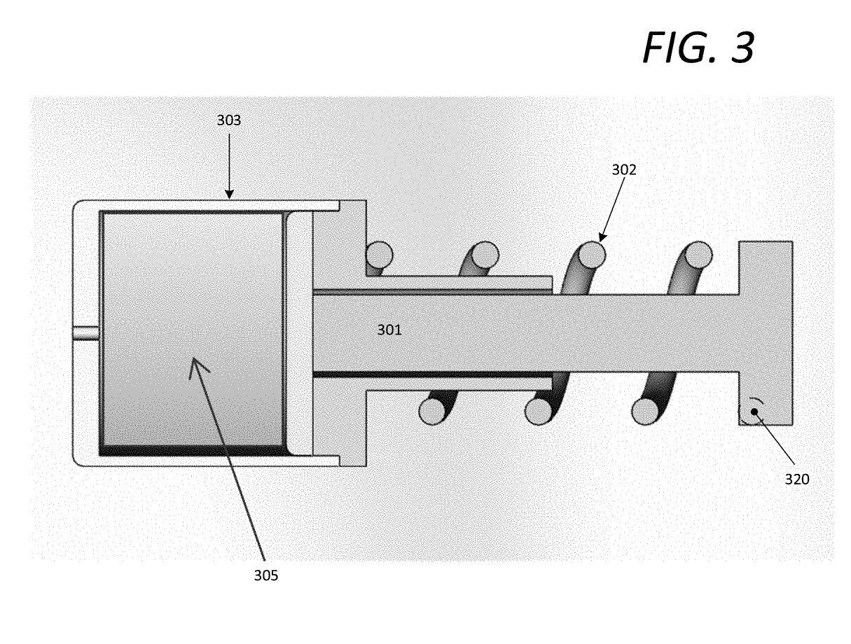

FIG. 3 shows the arrangement with plunger 301 retracted, similar to the representation of FIG. 1. SMA coil 302 is deformed and forced into an expanded orientation by the liquid 305 now within the formed reservoir. Note that in a typical arrangement, SMA coil 302 is joined in some manner to plunger 301, whether by fixed attachment to the right end of plunger 301, or at multiple points of contact with plunger 301. Circular contact point 320 represents the point where SMA coil 302 is fixedly mounted to plunger 301. Drawing liquid, such as liquid 305, into the opening of cylindrical tube 303, effectively “pulls” the SMA coil 302 into a second, “stretched” type shape as shown in FIG. 3.

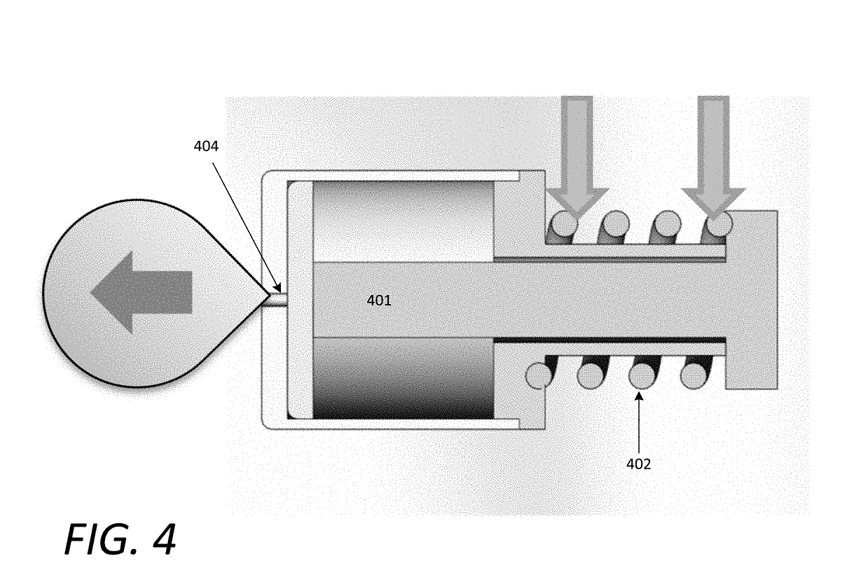

FIG. 4 illustrates application of power, such as by use of a power source and switch (not shown in this view). Application of power to SMA coil 402 causes the SMA coil to retract, thus compressing the plunger 401 into a compressed state and distributing liquid from nozzle 404. When power is applied to the SMA coil 402, it returns to its original shape or compresses, drawing plunger 401 toward nozzle 404, expelling liquid in the process.

Operation of an SMA (shape memory alloy) coil is generally known. An SMA coil is fabricated from SMA wire to form a coil that essentially “remembers” its original shape and when activated returns to that original shape. Different materials may be employed in an SMA wire used to make an SMA coil, including copper-aluminum-nickel, nickel-titanium, and alloys formed from zinc, copper, iron, and/or gold. In practice, a coil is made by wrapping SMA wire around a post or rod, where the distance between winding, thickness of the SMA wire, etc. determine the force applied when the resultant SMA coil is actuated. Such a coil is sometimes referred to as a “SMA spring coil.” Certain SMA coils can be actuated by different means, including heat and electricity, and while an electrical connection is shown in FIG. 1, it is to be appreciated that other types of actuation means may be employed.

When power is applied to the SMA coil 102 in FIG. 1, the coil is heated with electrical heat and the SMA coil 102 deforms and shrinks to the original shape, the SMA coil 102 pushes the plunger 101, and liquid is injected as shown in FIG. 4.

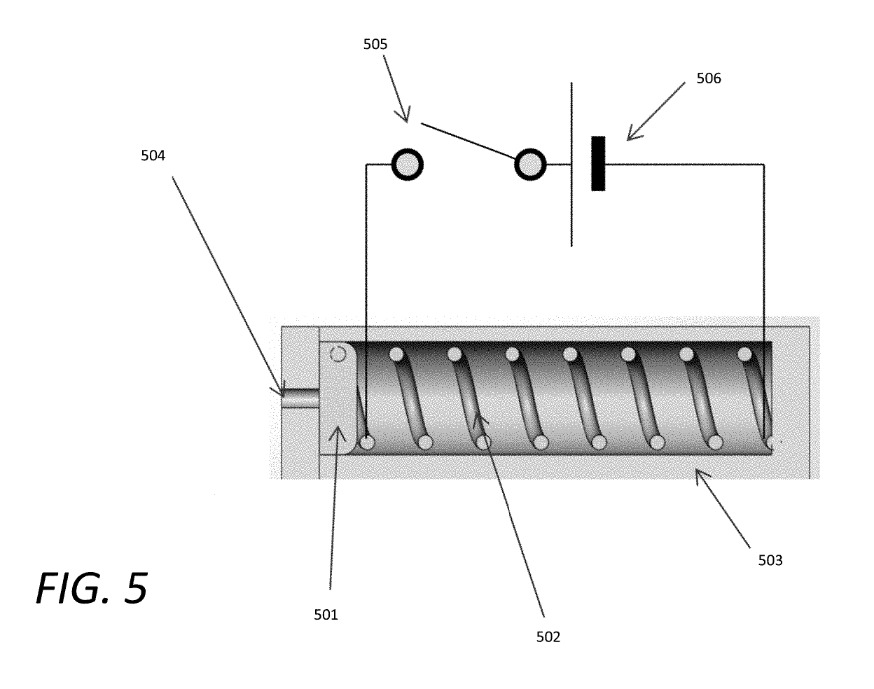

Alternately, the actuator device may be used as a collector. FIG. 5 illustrates an alternate version of the design used as a collector, including a plunger 501 formed without a central member and attached to SMA coil 502, in this view expanded. According to this embodiment, SMA coil 502 is located within cylindrical tube 503 and is attached to electronics, including switch 505 and power source 506, such that when power is applied and the SMA coil 502 heats up, the SMA coil 502 deforms and shrinks to an original shape. FIG. 5 illustrates the SMA coil 502 in an expanded orientation; application of electricity causes the SMA coil 502 to retract to an original position, thus pulling plunger 501 away from nozzle 504.

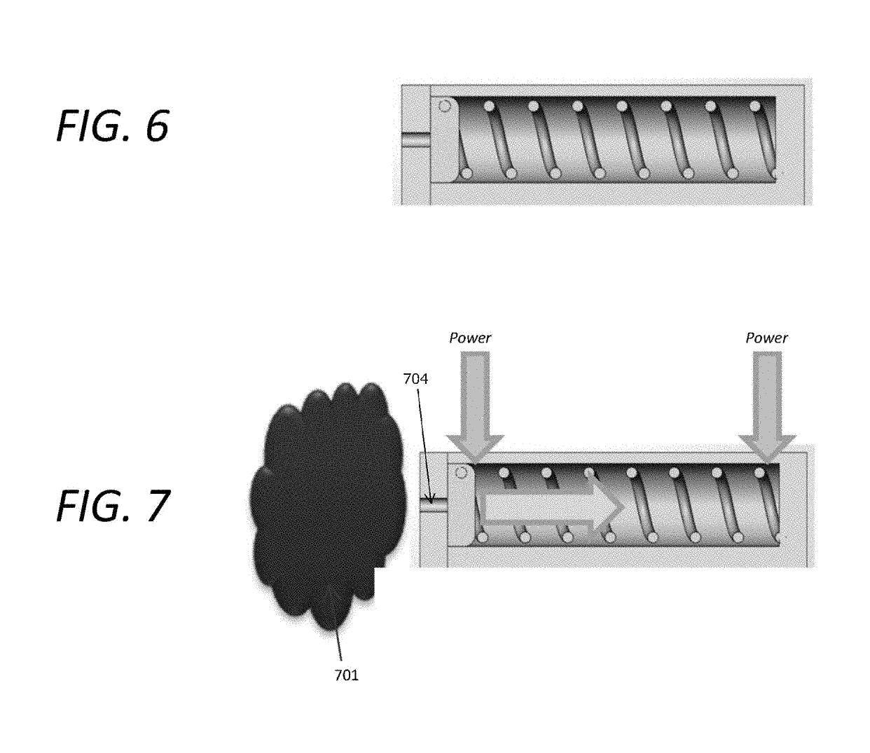

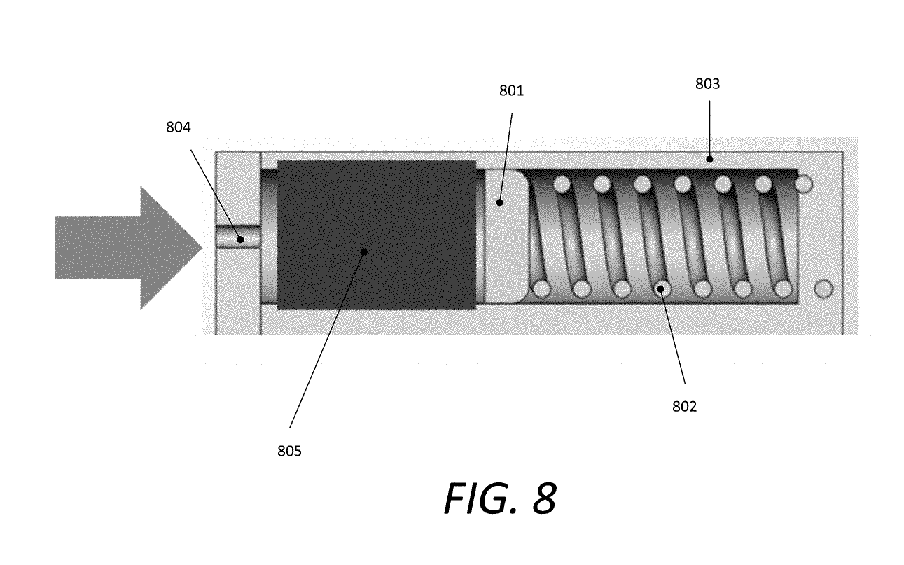

FIG. 6 illustrates the collector embodiment. FIG. 7 shows the collector embodiment with power attached, shown by the two arrows, with sample 701 available in front of the nozzle 704. FIG. 8 shows the result, with SMA coil 802 drawing the second embodiment plunger 801 away from nozzle 804, resulting in liquid 805 being drawn into second embodiment cylindrical tube 803. The region including liquid 805 may be called the reservoir.

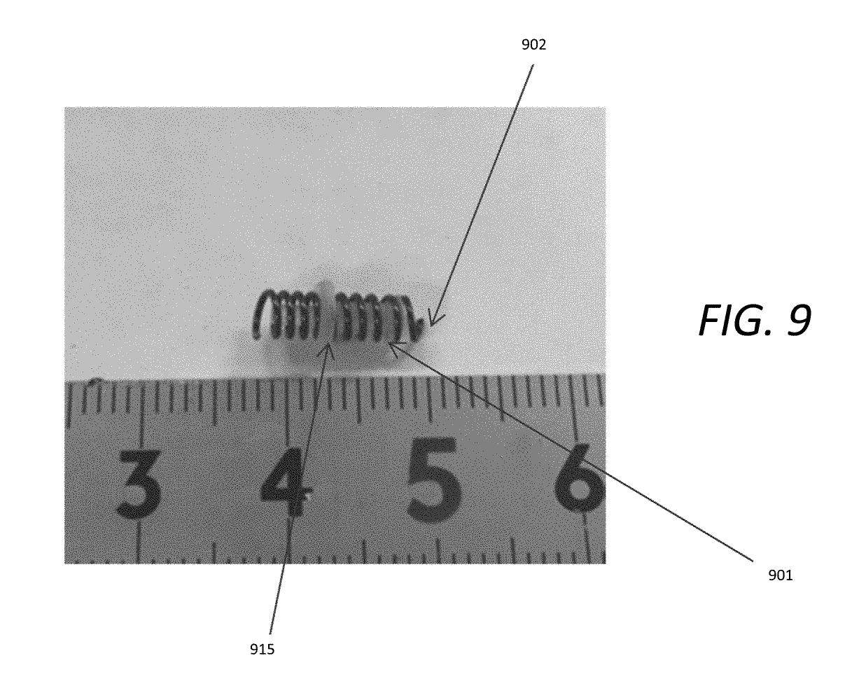

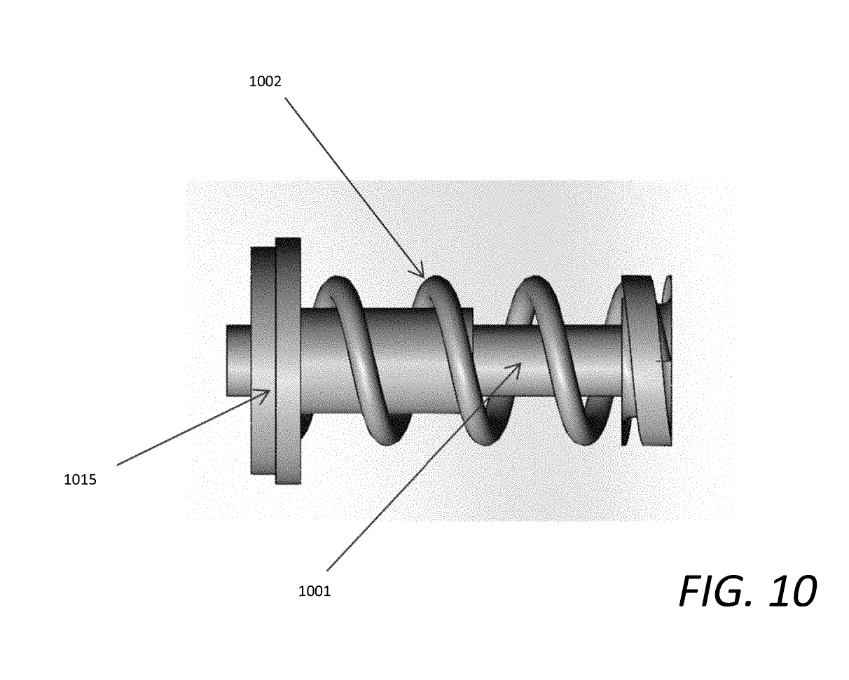

The syringe actuator can be small in size. FIG. 9 illustrates a SMA coil 902 on a plunger 901, having a cylindrical tube base 915. From FIG. 9, the SMA coil 902 may be approximately one inch in length approximately 9 or 10 turns. Other sizes and coil numbers may be employed. FIG. 10 is a conceptual version of the plunger including SMA coil 1002, plunger 1001, and cylindrical tube base 1015.

As may be appreciated from the various drawings presented, the SMA coil, such as SMA coil 1002, may be joined to the plunger or plunger cylindrical tube base, such as cylindrical tube base 1015, via a fixed mounting such that the SMA coil 1002 is formed within or fixedly joined to the plunger, or it may simply be placed on the exterior of the plunger to effectuate actuation without fixed mounting. In other words, the SMA coil may simply rest on the outside of the plunger and any cylindrical tube base and may push or provide force when the SMA coil is electrified or heated. The same is true of the end opposite the cylindrical tube base 1015 shown in FIG. 10. The far or remote end in any embodiment may be fixed to the plunger or associated hardware or may be free of the plunger or associated hardware depending on the implementation. For example, if the SMA coil is fully extended and application of electricity or heat causes the SMA coil to retract to draw in fluid, at least one end of the SMA coil must be affixed or fixedly mounted to the associated hardware to properly draw the plunger or component in a desired manner.

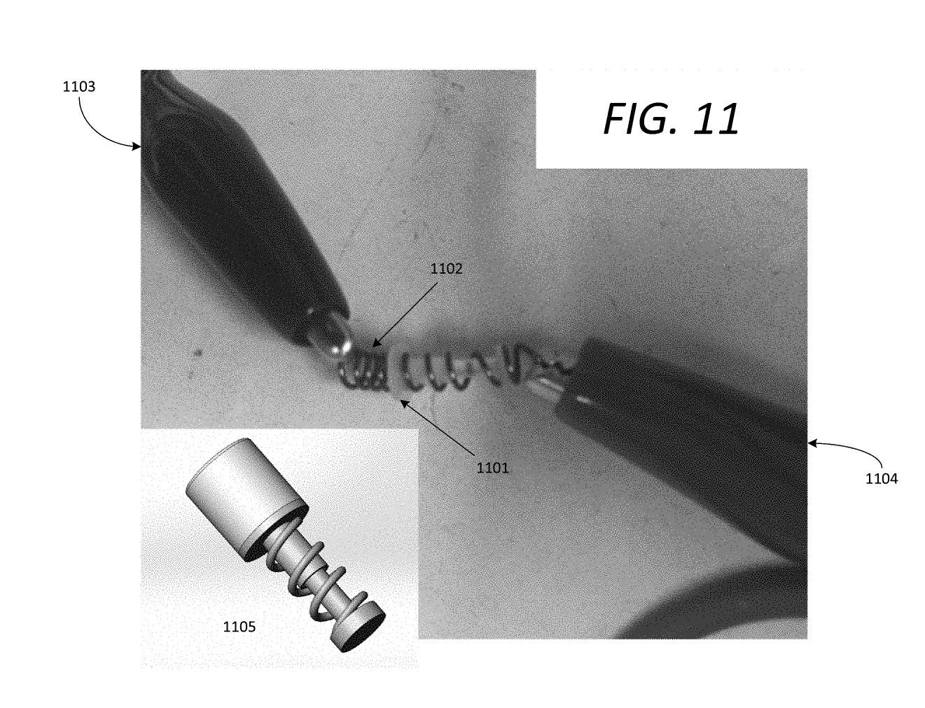

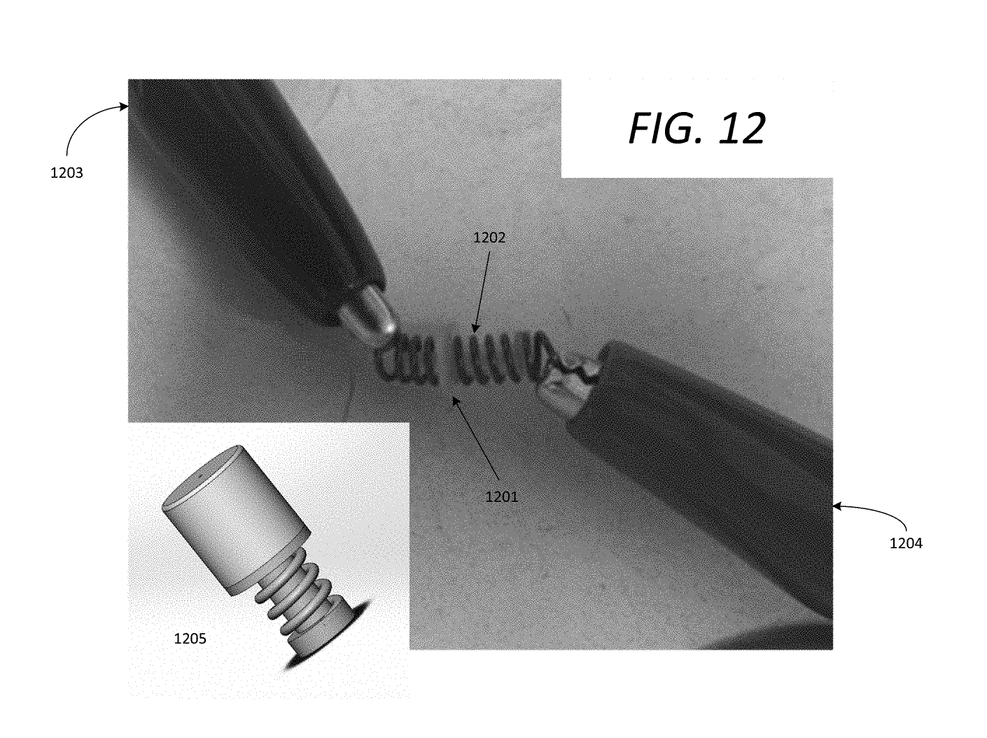

FIG. 11 shows SMA coil 1102 around plunger 1101 where SMA coil 1102 is connected to electrical connections 1103 and 1104. As shown by representation 1105, this drawing represents the uncompressed or extended version of the SMA coil. FIG. 12 shows the SMA coil 1202 around plunger 1201 where SMA coil 1202 is again connected to electrical connections 1203 and 1204. In this version, electricity has been applied and the SMA coil 1202 such that the SMA coil 1202 is heated to a compressed or retracted state as shown by representation 1205. When the electricity is turned off and heat no longer applied to the SMA coil 1202, the SMA coil 1202 returns to the form shown in FIG. 11.

In the foregoing representations, the syringe or plunger is driven using the SMA coil, and as a result the actuator can be small in size and weight without the need for complex and expensive actuation components. The actuator provides one way operation and can, if desired, be disposable. The design is relatively high efficiency, using a small amount of electrical power and the ability of the SMA coil to return to its original shape to drive the SMA coil and actuate the plunger. The foregoing device may be driven using a simple electrical circuit or heating arrangement.

One such application of the syringe actuator disclosed may employ an SMA coil formed of Flexinol by Dynalloy, Inc, and is the coil shown in FIG. 9. The coil alloy may be a Nickel-Titanium based alloy that reverts to approximately its original shape at 90 degrees Celsius. Coil dimensions may vary, but in one application the diameter of the wire may be on the order of 0.51 mm, coil outer diameter on the order of 3.45 mm, and original coil shape length on the order of 4.0 mm. The coil, when deformed, may attain a length of approximately 8.0 mm. Electricity may be applied to such a coil at 3 volts for approximately 10 seconds, whereby the coil reverts to its original shape and pushes the plunger.

Again, size and other parameters may vary, and larger implementations are possible as long as the SMA coil adequately drives the plunger.

The result of such a design is a low power, high efficiency injection arrangement that can provide liquid, such as a liquid drug, to an area or a patient with minimal power requirement and minimal complexity. The arrangement may vary in size, employing an SMA coil that may or may not be affixed to the plunger or parts associated with the plunger. In operation, liquid is provided, compressing the SMA coil, and electricity or heat provided to the SMA coil to expel the liquid from its reservoir.

Thus according to one embodiment, there is provided an actuator apparatus, comprising a slidable plunger comprising a remote end located proximate a reservoir within a vessel having an exit opening, a shape memory alloy (SMA) coil wound around the slidable plunger, and electronic circuitry connected to the SMA coil. Liquid may be provided to the reservoir via the exit opening, thereby sliding the slidable plunger away from the exit opening and expanding the SMA coil. When the SMA coil is expanded, electricity is provided via the electronic circuitry to the SMA coil to cause the SMA coil to return to a compressed configuration, moving the slidable plunger within the vessel to force the liquid from the vessel via the exit opening.

According to a second embodiment, there is provided an actuator apparatus, comprising a slidable actuation member located within a vessel having an exit opening, wherein the slidable actuation member positioned a distance from the exit opening forms a reservoir within the vessel, a shape memory alloy (SMA) coil positioned in association with the slidable actuation member to move the slidable actuation member within the vessel, and electronic circuitry connected to the SMA coil. Liquid may be provided to the reservoir via the exit opening, thereby moving the slidable actuation member away from the exit opening and compressing the SMA coil, and wherein when the SMA coil is thus compressed, electricity is provided via the electronic circuitry to the SMA coil to cause the SMA coil to return to an extended shape, pushing the slidable actuation member and expelling liquid from the vessel via the exit opening.

According to a further embodiment, there is provided an actuation method, comprising providing liquid through an exit opening to a reservoir in a vessel, thereby sliding a slidable actuation member located within the vessel away from the exit opening and concurrently compressing a shape memory alloy (SMA) coil positioned in association with the slidable actuation member, and applying electricity to the SMA coil via electronic circuitry. Applying electricity results in a production of heat, transitioning the SMA coil to an extended shape, thereby pushing liquid from the reservoir by pushing the slidable actuation member and expelling liquid through the exit opening.

The foregoing description of specific embodiments reveals the general nature of the disclosure sufficiently that others can, by applying current knowledge, readily modify and/or adapt the system and method for various applications without departing from the general concept. Therefore, such adaptations and modifications are within the meaning and range of equivalents of the disclosed embodiments. The phraseology or terminology employed herein is for the purpose of description and not of limitation.

What is claimed is:

1. An actuator apparatus, comprising:
a slidable plunger comprising a remote end located proximate a reservoir within a vessel having an exit opening;
a shape memory alloy (SMA) coil wound around the slidable plunger; and
electronic circuitry connected to the SMA coil;
wherein liquid may be provided to the reservoir via the exit opening, thereby sliding the slidable plunger away from the exit opening and expanding the SMA coil, and wherein when the SMA coil is expanded, electricity is provided via the electronic circuitry to the SMA coil to cause the SMA coil to return to a compressed configuration, moving the slidable plunger within the vessel to force the liquid from the vessel via the exit opening.

2. The actuator apparatus of claim 1, wherein the SMA coil is fixedly mounted to the slidable plunger.

3. The actuator apparatus of claim 1, wherein the electrical circuitry comprises a power source and a switch.

4. The actuator apparatus of claim 1, wherein the slidable plunger comprises a cylindrical tube base, and the SMA coil is fixedly mounted to the cylindrical tube base.

5. The actuator apparatus of claim 1, wherein the SMA coil is fixedly mounted to the vessel at a vessel end away from the exit opening.

6. The actuator apparatus of claim 1, wherein the SMA coil is formed from a nickel-titanium based alloy.

7. The actuator apparatus of claim 1, wherein liquid may be provided to the reservoir by:
drawing the slidable plunger away from the exit opening, thereby drawing the liquid into the reservoir, or
providing liquid through the exit opening using force.

8. An actuator apparatus, comprising:
a slidable actuation member located within a vessel having an exit opening, wherein the slidable actuation member positioned a distance from the exit opening forms a reservoir within the vessel;
a shape memory alloy (SMA) coil positioned in association with the slidable actuation member to move the slidable actuation member within the vessel; and
electronic circuitry connected to the SMA coil;
wherein liquid may be provided to the reservoir via the exit opening, thereby moving the slidable actuation member away from the exit opening and compressing the SMA coil, and wherein when the SMA coil is thus compressed, electricity is provided via the electronic circuitry to the SMA coil to cause the SMA coil to return to an extended shape, pushing the slidable actuation member and expelling liquid from the vessel via the exit opening.

9. The actuator apparatus of claim 8, wherein the SMA coil is fixedly mounted to the slidable actuation member.

10. The actuator apparatus of claim 8, wherein the electrical circuitry comprises a power source and a switch.

11. The actuator apparatus of claim 8, wherein the SMA coil is fixedly mounted to the vessel at a vessel end away from the exit opening.

12. The actuator apparatus of claim 8, wherein the SMA coil is formed from a nickel-titanium based alloy.

13. The actuator apparatus of claim 8, wherein liquid may be provided to the reservoir by providing liquid through the exit opening using force.

14. An actuation method, comprising:
providing liquid through an exit opening to a reservoir in a vessel, thereby sliding a slidable actuation member located within the vessel away from the exit opening and concurrently compressing a shape memory alloy (SMA) coil positioned in association with the slidable actuation member; and
applying electricity to the SMA coil via electronic circuitry;
wherein applying electricity results in application of heat to the SMA coil, transitioning the SMA coil to an extended shape, thereby pushing liquid from the reservoir by pushing the slidable actuation member and expelling liquid through the exit opening.

15. The actuation method of claim 14, wherein providing liquid through the exit opening comprises injecting the liquid through the exit opening using force.

16. The actuation method of claim 14, wherein the SMA coil is fixedly mounted to the slidable actuation member.

17. The actuation method of claim 14, wherein the electrical circuitry comprises a power source and a switch.

18. The actuation method of claim 14, wherein the SMA coil is fixedly mounted to the vessel at a vessel end away from the exit opening.

19. The actuation method of claim 14, wherein the SMA coil is formed from a nickel-titanium based alloy.
